(19) United States
(12) Patent Application Publication
(10) Pub. No.: US 2012/0157949 A1
(43) Pub. Date: Jun. 21, 2012

(54) Absorbent Article With Highlighted Active Barriers

(76) Inventors: Sandra Kay Knight, Omro, WI (US); Cheri Lee Schultz, Kimberly, WI (US); Andrew Mark Long, Appleton, WI (US); Jane Elizabeth Charbonneau, Menasha, WI (US)

(21) Appl. No.: 12/968,457

(22) Filed: Dec. 15, 2010

(51) Int. Cl. A61F 13/42

(52) U.S. Cl. 604/361

(57) ABSTRACT

An absorbent article for preventing leakage of a liquid insult includes an outer cover, a liner, and an absorbent assembly disposed therebetween, the liner having a body-facing surface; an active barrier disposed between the liner and the outer cover, wherein the active barrier is in fluid communication with the absorbent assembly, and wherein the active barrier is adapted to change dimensions when wetted; and a barrier highlight disposed to overlie the active barrier, wherein the barrier highlight is in fluid communication with the absorbent assembly or the active barrier, and wherein the barrier highlight is configured to change appearance when wetted.

### BACKGROUND

The present disclosure relates to absorbent articles that include a leakage prevention element. More specifically, the disclosure relates to an absorbent article such as feminine care products, incontinence products, and training pants that can provide both a barrier to potential leakage from the absorbent article and a noticeable physical sensation when the absorbent article is reaching fullness.

Absorbent articles such as feminine care products, incontinence products, and training pants are useful to absorb and contain body wastes. These products have developed to the extent that body exudates are quickly drawn and retained away from the wearer's skin so that the wearer remains relatively dry and comfortable. Although this improved performance enhances wearer dryness and comfort, it can reduce the wearer's ability to notice or recognize when the article is becoming full, especially if the wearer's attention is distracted by an activity. In one example, incontinence and feminine care product wearers are very concerned about leakage in public. Some wearers can be so bothered by leakage that if it occurs in a public place, they will avoid that place and situation for the rest of their life. Similar circumstances can apply to training pants and other disposable absorbent articles as well.

This application teaches products and methods to provide notice to a wearer that an active barrier is working to provide the wearer with confidence that leakage will not occur.

### SUMMARY

In response to the discussed deficiencies in the prior art, a new absorbent article has been developed. Absorbent articles of the present disclosure provide an active barrier against leakage, a physical sensation upon contact with urine or other body exudates, and a visual indication that the active barrier is working. As a result, the wearer or caregiver will notice distinct physical and visual sensations to assist the wearer or caregiver in recognizing when the absorbent article is nearing fullness.

In many cases, various elements of the articles described herein are white or otherwise uniform in their appearance and therefore difficult to differentiate. During use, even an active barrier can be difficult for a wearer or caregiver to recognize because of little color differentiation from the surrounding materials. Providing a highlighted region in the form of a barrier highlight that changes appearance under generally the same conditions that cause the active barrier to change dimensions provides a visual confirmation of and therefore confidence in product functionality. Consumers are thereby aware of the functional elements in the products and have a greater awareness of and confidence in the product performing as designed.

In one aspect of the present disclosure, an absorbent article for preventing leakage of a liquid insult includes an outer cover, a liner, and an absorbent assembly disposed therebetween, the liner having a body-facing surface; an active barrier disposed between the liner and the outer cover, wherein the active barrier is in fluid communication with the absorbent assembly, and wherein the active barrier is adapted to change dimensions when wetted; and a barrier highlight disposed to overlie the active barrier, wherein the barrier highlight is in fluid communication with the absorbent assembly or the active barrier, and wherein the barrier highlight is configured to change appearance when wetted.

In another aspect of the present disclosure, a feminine hygiene article for preventing leakage of a liquid insult includes an outer cover, a liner, and an absorbent assembly disposed therebetween, the liner having a body-facing surface, and the article including first and second transverse sides and a longitudinal direction; a first longitudinally-extending active barrier disposed between the liner and the outer cover and adjacent the first transverse side, wherein the active barrier is in fluid communication with the absorbent assembly, and wherein the active barrier is configured to change dimensions when wetted; and a barrier highlight disposed to overlie the active barrier, wherein the barrier highlight is in fluid communication with the absorbent assembly or the active barrier, and wherein the barrier highlight is configured to change appearance when wetted.

The purposes and features of the present disclosure will be set forth in the description that follows. Additional features of the disclosure can be realized and attained by the product and processes particularly pointed out in the written description and claims hereof, as well as from the appended drawings.

It is to be understood that both the foregoing general description and the following detailed description are exemplary and are intended to provide further explanation of the disclosure claimed. The accompanying drawings, which are incorporated in and constitute part of this specification, are included to illustrate and provide a further understanding of the disclosure.

### BRIEF DESCRIPTION OF THE DRAWINGS

The present disclosure will be more fully understood, and further features will become apparent, when reference is made to the following detailed description and the accompanying drawings. The drawings are merely representative and are not intended to limit the scope of the claims.

FIG. 1 representatively illustrates a plan view of a feminine/incontinence pad of the present disclosure showing the surface of the feminine/incontinence pad that faces the wearer when worn, and with portions cut away to show underlying features;

FIG. 2 representatively illustrates a perspective view of the feminine/incontinence pad of FIG. 1 including barriers;

FIG. 3 representatively illustrates a plan view of a pair of training pants of the present disclosure showing the surface of the training pants that faces the wearer when worn, and with portions cut away to show underlying features;

FIG. 4 representatively illustrates a plan view of an adult undergarment of the present disclosure showing the surface of the training pants that faces the wearer when worn, and with portions cut away to show underlying features;

FIGS. 5A and 5B representatively illustrate a schematic, cutaway, elevation view of a leakage prevention element used in conjunction with any of the articles of FIGS. 1-4;

FIGS. 6A and 6B representatively illustrate a schematic, cutaway, elevation view of an alternative leakage prevention element used in conjunction with any of the articles of FIGS. 1-4; and

FIG. 7 representatively illustrate a schematic plan view of another alternative leakage prevention element used in conjunction with any of the articles of FIGS. 1-4.

Repeat use of reference characters in the present specification and drawings is intended to represent the same or analogous features or elements of the present disclosure. The drawings are representational and are not necessarily drawn to scale. Certain proportions thereof can be exaggerated, while others can be minimized.

### DETAILED DESCRIPTION

Within the context of this specification, each term or phrase below includes the following meaning or meanings:

“Attach” and its derivatives refer to the joining, adhering, connecting, bonding, sewing together, or the like, of two elements. Two elements will be considered to be attached together when they are integral with one another or attached directly to one another or indirectly to one another, such as when each is directly attached to intermediate elements. “Attach” and its derivatives include permanent, releasable, or refastenable attachment. In addition, the attachment can be completed either during the manufacturing process or by the end wearer.

“Bond” and its derivatives refer to the joining, adhering, connecting, attaching, sewing together, or the like, of two elements. Two elements will be considered to be bonded together when they are bonded directly to one another or indirectly to one another, such as when each is directly bonded to intermediate elements. “Bond” and its derivatives include permanent, releasable, or refastenable bonding.

“Coform” refers to a blend of meltblown fibers and absorbent fibers such as cellulosic fibers that can be formed by air forming a meltblown polymer material while simultaneously blowing air-suspended fibers into the stream of meltblown fibers. The coform material can also include other materials, such as superabsorbent materials. The meltblown fibers and absorbent fibers are collected on a forming surface, such as provided by a foraminous belt. The forming surface can include a gas-pervious material that has been placed onto the forming surface.

“Connect” and its derivatives refer to the joining, adhering, bonding, attaching, sewing together, or the like, of two elements. Two elements will be considered to be connected together when they are connected directly to one another or indirectly to one another, such as when each is directly connected to intermediate elements. “Connect” and its derivatives include permanent, releasable, or refastenable connection. In addition, the connecting can be completed either during the manufacturing process or by the end wearer.

“Disposable” refers to articles that are designed to be discarded after a limited use rather than being laundered or otherwise restored for reuse.

The terms “disposed on,” “disposed along,” “disposed with,” or “disposed toward” and variations thereof are intended to mean that one element can be integral with another element, or that one element can be a separate structure bonded to or placed with or placed near another element.

“Elastic,” “elasticized,” “elasticity,” and “elastomeric” mean that property of a material or composite by virtue of which it tends to recover its original size and shape after removal of a force causing a deformation. Suitably, an elastic material or composite can be elongated by at least 50 percent (to 150 percent) of its relaxed length and will recover, upon release of the applied force, at least 40 percent of its elongation.

“Extensible” refers to a material or composite that is capable of extension or deformation without breaking, but does not substantially recover its original size and shape after removal of a force causing the extension or deformation. Suitably, an extensible material or composite can be elongated by at least 50 percent (to 150 percent) of its relaxed length.

“Fiber” refers to a continuous or discontinuous member having a high ratio of length to diameter or width. Thus, a fiber can be a filament, a thread, a strand, a yarn, or any other member or combination of these members.

“Hydrophilic” describes fibers or the surfaces of fibers that are wetted by aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by a Cahn SFA-222 Surface Force Analyzer System, or a substantially equivalent system. When measured with this system, fibers having contact angles less than 90 degrees are designated “wettable” or hydrophilic, and fibers having contact angles greater than 90 degrees are designated “nonwettable” or hydrophobic.

“Join” and its derivatives refer to the connecting, adhering, bonding, attaching, sewing together, or the like, of two elements. Two elements will be considered to be joined together when they are integral with one another or joined directly to one another or indirectly to one another, such as when each is directly joined to intermediate elements. “Join” and its derivatives include permanent, releasable, or refastenable joinder. In addition, the joining can be completed either during the manufacturing process or by the end wearer.

“Layer” when used in the singular can have the dual meaning of a single element or a plurality of elements.

“Liquid impermeable,” when used in describing a layer or multi-layer laminate means that liquid, such as urine, will not pass through the layer or laminate, under ordinary use conditions, in a direction generally perpendicular to the plane of the layer or laminate at the point of liquid contact.

“Liquid permeable” refers to any material that is not liquid impermeable.

“Meltblown” refers to fibers formed by extruding a molten thermoplastic material through a plurality of fine, usually circular, die capillaries as molten threads or filaments into converging high velocity gas (e.g., air) streams, generally heated, which attenuate the filaments of molten thermoplastic material to reduce their diameters. Such a process is disclosed, for example, in U.S. Pat. No. 3,849,241 to Butin et al. Meltblown fibers can be continuous or discontinuous and are generally self bonding when deposited onto a collecting surface.

“Member” when used in the singular can have the dual meaning of a single element or a plurality of elements.

“Nonwoven” and “nonwoven web” refer to materials and webs of material that are formed without the aid of a textile weaving or knitting process. For example, nonwoven materials, fabrics or webs have been formed from many processes such as, for example, meltblowing processes, spunbonding processes, air laying processes, and bonded carded web processes.

“Stretchable” means that a material can be stretched, without breaking, by at least 50 percent (to 150 percent of its initial (unstretched) length) in at least one direction. Elastic materials and extensible materials are each stretchable materials.

“Superabsorbent material” refers to a water-swellable, water-insoluble organic or inorganic material capable, under the most favorable conditions, of absorbing at least about ten times its weight and, more desirably, at least about thirty times its weight in an aqueous solution containing about 0.9 weight percent sodium chloride.

These terms can be defined with additional language in the remaining portions of the specification.

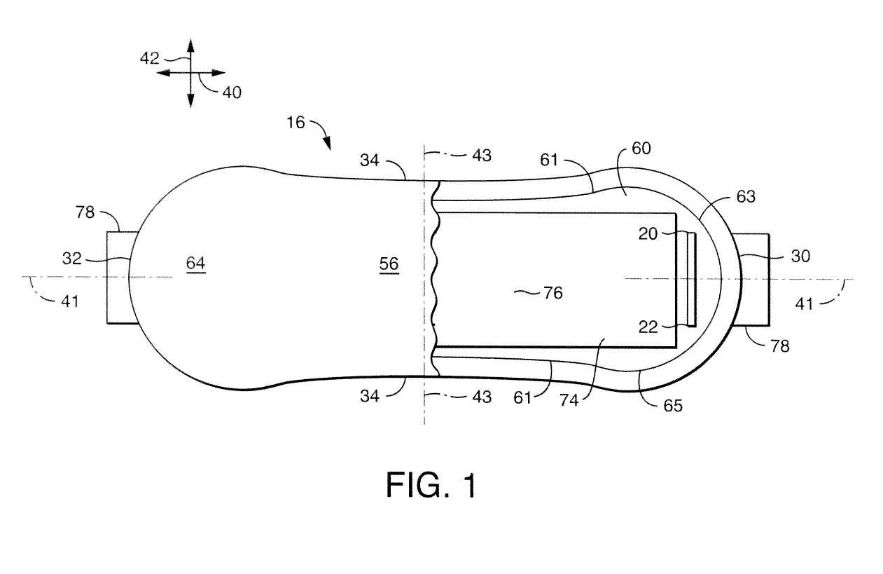

A leakage prevention element 20 includes an active barrier 22. While a leakage prevention element 20 is illustrated in FIG. 1 with a feminine/incontinence pad 16, the leakage prevention element 20 can also be used in conjunction with other garments. For example, a leakage prevention element 20 of the disclosure can be used with other disposable absorbent articles such as diapers, diaper pants, incontinence articles, feminine pads, and liners, or the like. The descriptions of the various absorbent articles 10 described herein are for exemplary purposes only. Variations in the structures, materials, and designs of the absorbent articles 10 that do not impact the subject matter of this disclosure are possible and expected.

Referring now to the drawings and in particular to FIG. 1, an absorbent article 10 of the present disclosure is representatively illustrated in the form of a feminine/incontinence pad and is indicated in its entirety by the reference numeral 16. The exemplary feminine/incontinence pad 16 includes an outer cover (otherwise referred to as a baffle or backsheet, not shown), an absorbent assembly 60, an optional tissue layer 74, an optional liquid acquisition and distribution layer 76 and a bodyside liner 64 (also referred to as the topsheet). The feminine/incontinence pad 16 also has first and second side edges 34 that are the longitudinal sides of the elongated feminine/incontinence pad 16. The side edges 34 can be contoured, for example, in a concave shape, or they can be linear. The feminine/incontinence pad 16 has a longitudinal direction 40 and a transverse direction 42, and includes a longitudinal centerline 41 and a transverse centerline 43.

The side edges 34 can further include flaps (not shown) that extend laterally outward. Flaps are known in the art and are shown in, for example U.S. Pat. No. 6,387,084 issued to VanGompel et al., the contents of which are incorporated herein by reference to the extent that they are consistent (i.e., not in conflict) herewith. In one aspect (not shown), one or more elastic elements are disposed along the sides to form a gasket with the body of the wearer. Elastic sides are known in the art, as is shown in U.S. Pat. No. 6,315,765 issued to Datta et al., the contents of which are incorporated herein by reference to the extent that they are consistent (i.e., not in conflict) herewith. In one aspect, the elastic elements are disposed between the bodyside liner 64 and the outer cover.

The feminine/incontinence pad 16 has a bodyside inner surface 56 and a garment-side outer surface. Applied to at least a portion of the garment-side outer surface is a garment attachment adhesive. In various aspects, the garment attachment adhesive is configured as a single band of adhesive or as two or more spaced apart strips. Alternatively, the garment attachment adhesive can include a swirl pattern of adhesive that encompasses a major portion of the garment-side outer surface of the feminine/incontinence pad 16.

A release strip 78, also known as a releasable peel strip, is removably secured to the garment attachment adhesive and serves to prevent premature contamination of the adhesive before the feminine/incontinence pad 16 is secured to the crotch portion of an undergarment. In various aspects, the garment attachment adhesive is designed to be secured to the inner crotch portion of an undergarment so as to keep the absorbent product in register with the body of the wearer. The release strip 78 can extend beyond one or both of the end edges 30, 32 of the outer cover, as shown in FIG. 1. Alternatively, the release strip 78 can be as short as the length of the garment attachment adhesive, or slightly longer than the adhesive or can be only as long as the garment attachment adhesive, but does not extend beyond the end edges 30, 32 of the outer cover.

The bodyside liner or topsheet 64, which is preferably liquid permeable, can be formed from one or more materials. The bodyside liner or topsheet 64 must be able to manage different body excretions depending on the type of product. In feminine care products, often the bodyside liner or body-contacting layer 64 must be able to handle menses and urine. In the present disclosure, the bodyside liner or topsheet 64 can include a layer constructed of any operative material, and can be a composite material. For example, the bodyside liner 64 can include a woven fabric, a nonwoven fabric, a polymer film, a film-nonwoven fabric laminate or the like, as well as combinations thereof. Examples of a nonwoven fabric useable in the bodyside liner 64 include, for example, an airlaid nonwoven web, a spunbond nonwoven web, a meltblown nonwoven web, a bonded-carded-web, a hydroentangled nonwoven web, a spunlace web or the like, as well as combinations thereof. Other examples of suitable materials for constructing the bodyside liner 64 can include rayon, bonded-carded-webs of polyester, polypropylene, polyethylene, nylon, or other heat-bondable fibers finely perforated film webs, net-like materials, and the like, as well as combinations thereof. These webs can be prepared from polymeric materials such as, for example, polyolefins, such as polypropylene and polyethylene and copolymers thereof, polyesters in general including aliphatic esters such as polylactic acid, nylon or any other heat-bondable materials.

Other examples of suitable materials for the bodyside liner or topsheet 64 are composite materials of a polymer and a nonwoven fabric material. The composite materials are typically in the form of integral sheets generally formed by the extrusion of a polymer onto a nonwoven web, such as a spunbond material. In a desired arrangement, the liner 64 can be configured to be operatively liquid-permeable with regard to the liquids that the article is intended to absorb or otherwise handle. The operative liquid-permeability can, for example, be provided by a plurality of pores, perforations, apertures or other openings, as well as combinations thereof, which are present or formed in the liner or body contacting layer. The apertures or other openings can help increase the rate at which bodily liquids can move through the thickness of the liner or body contacting layer and penetrate into the other components of the article (e.g. into the absorbent assembly 60). The selected arrangement of liquid-permeability is desirably present at least on an operative portion of the bodyside liner 64 that is appointed for placement on the body-facing side of the article. The bodyside liner 64 can provide comfort and conformability, and can function to direct bodily exudates away from the body and toward the absorbent assembly 60. The bodyside liner 64 can be configured to retain little or no liquid in its structure, and can be configured to provide a relatively comfortable and non-irritating surface next to the body tissues of a wearer. In the present disclosure, the topsheet or body-facing surface of each absorbent article 10 can be embossed, printed, or otherwise imparted with a pattern.

The outer cover can include a layer constructed of any operative material, and can have a selected level of liquid-permeability or liquid-impermeability, as desired. In a particular configuration, the outer cover can be configured to provide an operatively liquid-impermeable baffle structure. The outer cover can, for example, include a polymeric film, a woven fabric, a nonwoven fabric or the like, as well as combinations or composites thereof. For example, the outer cover can include a polymer film laminated to a woven or nonwoven fabric. In a particular feature, the polymer film can be composed of polyethylene, polypropylene, polyester or the like, as well as combinations thereof. Additionally, the polymer film can be micro-embossed, have a printed design, have a printed message to the consumer, and/or can be at least partially colored. Suitably, the outer cover can operatively permit a sufficient passage of air and moisture vapor out of the article, particularly out of an absorbent (e.g. storage or absorbent assembly 60) while blocking the passage of bodily liquids. An example of a suitable outer cover material can include a breathable, microporous film, such as those described in, for example, U.S. Pat. No. 6,045,900 to McCormack et al.

Bicomponent films or other multi-component films can also be used, as well as woven and/or nonwoven fabrics that have been treated to render them operatively liquid-impermeable. Another suitable outer cover material can include a closed cell polyolefin foam. For example, a closed-cell polyethylene foam can be employed.

The liquid-permeable bodyside liner 64 and the liquid-impermeable outer cover can be peripherally sealed together to enclose the absorbent assembly 60 to form the feminine/incontinence pad 16. Alternatively, the bodyside liner 64 can be wrapped around both the absorbent assembly 60 and the outer cover to form a wrapped pad. The bodyside liner 64 and outer cover, and other components of the feminine/incontinence pad 16, can be joined for example with adhesive bonds, sonic bonds, thermal bonds, pinning, stitching or any other attachment techniques known in the art, as well as combinations thereof.

The absorbent assembly 60 can be in a variety of shapes and configurations as are known in the art, such as rectangular, hourglass shaped, l-shaped, and the like. The absorbent assembly 60 has opposed lateral edges 61 and opposed longitudinal ends 63. The lateral edges 61 and longitudinal ends 63 together make up the perimeter 65 of the absorbent assembly 60.

The absorbent assembly 60 is designed to absorb body exudates, including menstrual fluid, blood, urine, and other body fluids. The absorbent assembly 60 can contain one or more layers of absorbent material. The layers can contain similar materials or different materials. Suitable materials for the absorbent assembly 60 include, for example, cellulose, wood pulp fluff, rayon, cotton, and meltblown polymers such as polyester, polypropylene or coform. Coform is a meltblown air-formed combination of meltblown polymers, such as polypropylene, and absorbent staple fibers, such as cellulose. A preferred material is wood pulp fluff, for it is low in cost, relatively easy to form, and has good absorbency.

The absorbent assembly 60 can also be formed from a composite including a hydrophilic material that can be formed from various natural or synthetic fibers, wood pulp fibers, regenerated cellulose or cotton fibers, or a blend of pulp and other fibers. A desired material is an airlaid material.

In one aspect, the absorbent assembly 60 also includes a superabsorbent material, in addition to or in place of the hydrophilic material, which increases the ability of the absorbent assembly 60 to absorb a large amount of fluid in relation to its own weight. Generally stated, the superabsorbent material can be a water-swellable, generally water-insoluble, hydrogel-forming polymeric absorbent material, which is capable of absorbing at least about 15, suitably about 30, and possibly about 60 times or more its weight in physiological saline (e.g. saline with 0.9 wt % NaCl). The superabsorbent materials can be inserted as particles or in sheet form. The superabsorbent material can be biodegradable or bipolar. The hydrogel-forming polymeric absorbent material can be formed from organic hydrogel-forming polymeric material, which can include natural material such as agar, pectin, and guar gum; modified natural materials such as carboxymethyl cellulose, carboxyethyl cellulose, and hydroxypropyl cellulose; and synthetic hydrogel-forming polymers. Synthetic hydrogel-forming polymers include, for example, alkali metal salts of polyacrylic acid, polyacrylamides, polyvinyl alcohol, ethylene maleic anhydride copolymers, polyvinyl ethers, polyvinyl morpholinone, polymers and copolymers of vinyl sulfonic acid, polyacrylates, polyacrylamides, polyvinyl pyridine, and the like. Other suitable hydrogel-forming polymers include hydrolyzed acrylonitrile grafted starch, acrylic acid grafted starch, and isobutylene maleic anhydride copolymers and mixtures thereof. The hydrogel-forming polymers can be lightly crosslinked to render the material substantially water insoluble. Crosslinking can, for example, be by irradiation or covalent, ionic, Van der Waals, or hydrogen bonding. Hydroxyfunctional polymers have been found to be good superabsorbents for sanitary napkins. Such superabsorbents are commercially available from Evonik Stockhausen, LLC, Greensboro, N.C., U.S.A.; BASF Corporation, Florham Park, N.J., U.S.A.; and Nippon Shokubai, Chattanooga, Tenn., U.S.A., among others, and are a partially neutralized salt of cross-linked copolymer of polyacrylic acid and polyvinyl alcohol having an absorbency under load value above 25 grams of absorbed liquid per gram of absorbent material (g/g). Other types of superabsorbent materials known to those skilled in the art can also be used.

Additional layers or substrates, including for example, the liquid acquisition and distribution layer 76, and an optional tissue layer 74 can also be incorporated into the feminine/incontinence pad 16.

The leakage prevention element 20 is positioned within the feminine/incontinence pad 16 so that urine or other body exudates filling the absorbent assembly 60 contacts the leakage prevention element 20 prior to completely filling and eventually leaking from the feminine/incontinence pad 16. Thus, the leakage prevention element 20 is disposed with or near the absorbent assembly 60 so that urine or other body exudates contacting the absorbent assembly 60 will also eventually contact the leakage prevention element 20. Most desirably, the leakage prevention element 20 is disposed on the bodyside of the absorbent assembly 60 so as to be sandwiched between the absorbent assembly 60 and the bodyside liner 64. In this way, the physical sensation resulting from the leakage prevention element 20 is more easily noticed by the wearer.

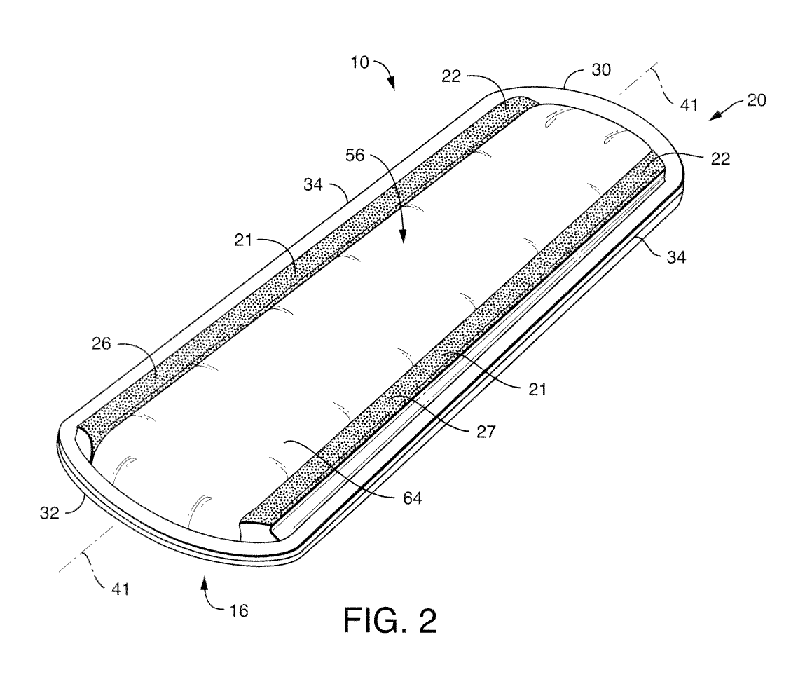

In one aspect of the present disclosure, the leakage prevention element 20 can include barrier structures 26 and 27 to prevent body fluids from reaching the sides 34 of the feminine/incontinence pad 16, as illustrated in FIG. 2. The barrier structures 26 and 27 are longitudinally positioned between the longitudinal centerline 41 and the sides 34 of the feminine/incontinence pad 16 and preferably, are positioned parallel to the longitudinal centerline 41. By providing longitudinal barriers 26 and 27 inward of the sides 34, the lateral migration of body fluid insulting the bodyside liner 64 can be directed longitudinally along the barriers 26 and 27. Thus, greater utilization of the absorbent assembly 60 is achieved, lessening the likelihood of the feminine/incontinence pad 16 leaking from the sides 34.

One of the barriers 26, 27 is positioned laterally to the longitudinal centerline 41, between the longitudinal centerline 41 and the side 34 of the feminine/incontinence pad 16. The barrier 26 or 27 can reside on top of the bodyside liner 64 or desirably, is positioned between the bodyside liner 64 and the absorbent assembly 60. In a preferred aspect, the barrier 26 or 27 is positioned between the bodyside liner 64 and the absorbent assembly 60, and adjacent a first longitudinal side edge 34. This arrangement facilitates halting the lateral flow of absorbed liquids toward the side 34 through the absorbent assembly 60 as well as unabsorbed liquids across the bodyside liner 64. The barrier 26 or 27 can have a linear shape of a line or can be curved while remaining in the area between the longitudinal centerline 41 and the side 34. Desirably, each barrier 26, 27 is adjacent to an edge 34 following the contour of the absorbent assembly 60. The barriers 26, 27 can extend at least 10% of the length of the absorbent assembly 60. The barriers 26, 27 can otherwise extend over substantially the full length of the absorbent assembly 60.

As shown in FIG. 2, barrier elements 26 and 27 extend upwardly. The barriers 26, 27 can have a dry width ranging from about 3 millimeters to about 12 millimeters and preferably from about 3 millimeters to about 8 millimeters. The barriers 26, 27 can have a dry height ranging from about 2 millimeters to about 25 millimeters; preferably, from about 6 millimeters to about 15 millimeters; and most preferably, the barriers 26, 27 each has a dry height extending above the plane of the absorbent body-facing surface.

In another aspect of the present disclosure (not shown), the leakage prevention element 20 can be located within the absorbent assembly 60 or beneath the absorbent assembly 60. The leakage prevention element 20 can also be positioned on the flaps or in any other suitable position in the feminine/incontinence pad 16, as long as fluid communication is provided between the absorbent assembly 60 and the leakage prevention element 20. In addition, leakage prevention elements 20 can be positioned in more than one location within the feminine/incontinence pad 16. The leakage prevention element 20 can be maintained in position by bonding, using adhesives, ultrasonic bonds, or other suitable means.

One or more leakage prevention elements 20 can be disposed in the feminine/incontinence pad 16. A pair of leakage prevention elements 20 can be positioned on opposite sides of the longitudinal centerline 41 and spaced apart from the intersection of the longitudinal and transverse centerlines 41, 43 along the transverse centerline 43. Similarly, a pair of leakage prevention elements 20 can be positioned on opposite sides of the transverse centerline 43 and spaced apart from the intersection of the longitudinal and transverse centerlines 41, 43 along the longitudinal centerline 41. In another aspect, leakage prevention elements 20 can be positioned at each of the points at which a centerline meets the sides 34 of the absorbent assembly 60. In still another aspect, the leakage prevention elements 20 can be positioned completely or partially along the entire absorbent assembly perimeter 65. In yet another aspect, the leakage prevention elements 20 can be positioned completely or partially in a spaced-apart manner from the absorbent assembly perimeter 65.

The position and/or structure of the leakage prevention elements 20 should be such that the leakage prevention elements 20 come in contact with urine or other bodily waste as the absorbent assembly 60 fills, but prior to any leakage from the absorbent assembly 60. The leakage prevention elements 20 can be centered in the longitudinal direction 40. Alternatively, however, the leakage prevention elements 20 can be located off the transverse centerline 43 of the feminine/incontinence pad 16. Likewise, the leakage prevention elements 20 can be centered in the transverse direction 42 or can be located off the longitudinal centerline 41 of the feminine/incontinence pad 16.

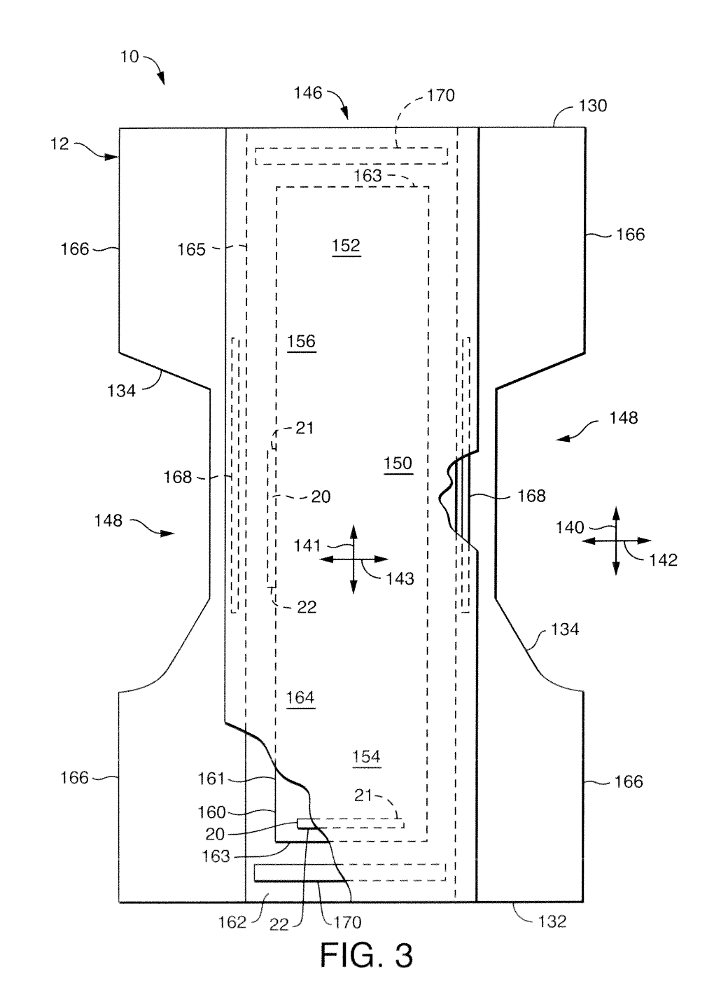

Referring now to the drawings and in particular to FIG. 3, an absorbent article 10 of the present disclosure is representatively illustrated in the form of children's toilet training pants and is indicated in its entirety by the reference numeral 12. Absorbent articles 10 of this type are described in more detail in U.S. Pat. No. 5,681,298 issue to Brunner et al., the contents of which are incorporated herein by reference to the extent that they are consistent (i.e., not in conflict) herewith. The absorbent article 10 includes a leakage prevention element 20 that is adapted to block leakage from the article 10 and to create a distinct physical sensation to the wearer upon the absorbent article 10 nearing fullness, which can enhance a wearer's ability to recognize when leakage can be a threat.

With reference to FIG. 3, an absorbent article 10 formed according to the disclosure is shown for purposes of illustration as a disposable training pant 12 for use by a child. The training pant 12 includes a leakage prevention element 20 that is positioned and adapted to create a distinct physical sensation as the training pant 12 approaches fullness. Because the physical sensation is noticeable to the child, the child's ability to recognize when fullness is occurring will be enhanced. The training pant 12 will now be described in greater detail.

The training pant 12 is illustrated at an intermediate stage of assembly and in a flat and stretched condition in FIG. 3. The training pant 12 has opposite longitudinally spaced front and back end edges 130 and 132, and opposite side edges 134 extending between the end edges. The training pant 12 also defines longitudinal and transverse directions represented by arrows 140 and 142 in FIG. 3. The training pant 12 includes a longitudinal centerline 141 and a transverse centerline 143.

The finished training pant 12 becomes three-dimensional and thus defines a waist opening 146 and two leg openings 148 (FIG. 3). The finished training pant 12 has a crotch region 150 generally located between the leg openings 148. The crotch region 150 includes that portion of the training pant 12 that, when worn, is positioned between the legs of the wearer and covers the lower torso of the wearer. A front waist region 152 of the training pant 12 extends generally from the crotch region 150 to the front end edge 130, and a back waist region 154 extends from the crotch region 150 to the back end edge 132. In general, the longitudinal extent of the waist regions 152, 154 is related to the distance between the end edges 130 and 132 of the training pant 12 and the crotch region 150, measured along the side edges 134. The training pant 12 also includes an inner surface 156 and an opposite outer surface (not shown).

The illustrated training pant 12 includes an absorbent assembly 160 sandwiched between an outer cover 162 and a bodyside liner 164. The outer cover 162 and liner 164 are desirably longer and wider than the absorbent assembly 160 and bonded together using adhesives, thermal bonds, ultrasonic bonds or other suitable means. Further, the absorbent assembly 160 is disposed on the outer cover 162, and can be bonded directly thereto using adhesives, thermal bonds, ultrasonic bonds or other suitable means. The liner 164 can be bonded directly to the absorbent assembly 160 as well.

The outer cover 162 can, for instance, include a single layer of film, a woven material, a nonwoven material or another suitable liquid permeable or liquid impermeable material. The outer cover 162 can include a thin, substantially liquid impermeable web or sheet of plastic film such as polyethylene, polypropylene, polyvinyl chloride or similar material. Alternatively, the outer cover 162 can include a nonwoven, fibrous web that has been suitably constructed and arranged to be substantially liquid impermeable. Still alternatively, the outer cover 162 can include a layered or laminated material, such as a thermally bonded plastic film and nonwoven web composite or a stretch bonded laminate.

The outer cover 162 can suitably include a material that is substantially liquid impermeable. The outer cover 162 can be provided by a single layer of liquid impermeable material, or more suitably include a multi-layered laminate structure in which at least one of the layers is liquid impermeable. In particular aspects, the outer layer can suitably provide a relatively cloth-like texture to the wearer. A suitable liquid impermeable film for use as a liquid impermeable inner layer, or a single layer liquid impermeable outer cover 162 is a 0.025 millimeter (1.0 mil) polyethylene film commercially available from Edison Plastics Inc. of South Plainfield, N.J., U.S.A. Alternatively, the outer cover 162 can include a woven or non-woven fibrous web layer that has been totally or partially constructed or treated to impart the desired levels of liquid impermeability to selected regions that are adjacent or proximate the absorbent assembly.

The outer cover 162 can also be stretchable, and in some aspects it can be elastomeric. For example, such an outer cover material can include a 0.3 osy polypropylene spunbond that is necked 60 percent in the transverse direction 142 and creped 60 percent in the longitudinal direction 140, laminated with 3 grams per square meter (gsm) Bostik-Findley H2525A styrene-isoprene-styrene based adhesive to 8 gsm PEBAX 2533 film with 20 percent TiO₂ concentrate. Reference is made to U.S. Pat. No. 5,883,028, issued to Morman et al., U.S. Pat. No. 5,116,662 issued to Morman and U.S. Pat. No. 5,114,781 issued to Morman, the contents of which are hereby incorporated herein by reference to the extent that they are consistent (i.e., not in conflict) herewith, for additional information regarding suitable outer cover materials.

The bodyside liner 164 can be any soft, flexible, porous sheet that passes liquids therethrough. The liner 164 can include, for example, a nonwoven web or sheet of wet strength tissue paper, a spunbonded, meltblown or bonded-carded web composed of synthetic polymer filaments, such as polypropylene, polyethylene, polyesters or the like, or a web of natural polymer filaments such as rayon or cotton. The liner 164 has a pore size that readily allows the passage therethrough of liquids, such as urine and other body exudates. The liner 164 can be selectively embossed or perforated with discrete slits or holes extending therethrough. Optionally, the web or sheet can be treated with a surfactant to aid in liquid transfer. One suitable liner material is a wettable spunbonded polypropylene web produced by the methods and apparatus described in U.S. Pat. Nos. 4,340,563 issued Jul. 20, 1982, and 4,405,297 issued Sep. 23, 1983, to Appel et al., the contents of which are incorporated herein by reference to the extent that they are consistent (i.e., not in conflict) herewith.

Alternatively, the bodyside liner 164 can also be stretchable, and in some aspects it can be elastomeric. For instance, the liner 164 can be a non-woven, spunbond polypropylene fabric composed of about 2 to 3 denier fibers formed into a web having a basis weight of about 12 gsm that is necked approximately 60 percent. Strands of about 9 gsm KRATON G2760 elastomer material placed eight strands per inch (2.54 cm) can be adhered to the necked spunbond material to impart elasticity to the spunbond fabric. The fabric can be surface treated with an operative amount of surfactant, such as about 0.6 percent AHCOVEL Base N62 surfactant, available from ICI Americas of Wilmington, Del., U.S.A. Other suitable materials can be extensible biaxially stretchable materials, such as a neck stretched/creped spunbond. Reference is made to U.S. Pat. No. 6,552,245, issued Apr. 22, 2003, to Roessler et al., the contents of which are incorporated herein by reference to the extent that they are consistent (i.e., not in conflict) herewith.

The absorbent assembly 160 can be in a variety of shapes and configurations as are known in the art, such as rectangular, hourglass shaped, l-shaped, and the like. The absorbent assembly 160 has opposed lateral edges 161 and opposed longitudinal ends 163. The lateral edges 161 and longitudinal ends 163 together make up the perimeter 165 of the absorbent assembly 160.

The absorbent assembly 160 can include various absorbent materials, such as an air-formed batt of cellulosic fibers (i.e., wood pulp fluff) or a coform material composed of a mixture of cellulosic fibers and synthetic polymer fibers. The absorbent assembly 160 can also include compounds to increase its absorbency, such as 0-95 weight percent of organic or inorganic high-absorbency materials, which are typically capable of absorbing at least about 15 and desirably more than 25 times their weight in water. Suitable high-absorbency materials are described in U.S. Pat. No. 4,699,823 issued Oct. 13, 1987, to Kellenberger et al. and U.S. Pat. No. 5,147,343 issued Sep. 15, 1992, to Kellenberger, the contents of which are incorporated herein by reference to the extent that they are consistent (i.e., not in conflict) herewith. High-absorbency materials are available from various commercial vendors. The absorbent assembly 160 can also include tissue layers or acquisition or distribution layers to help maintain the integrity of fibrous absorbents or transport liquids (not shown).

The absorbent assembly 160 is suitably compressible, conformable, and capable of absorbing and retaining liquid body exudates released by the wearer. For example, the absorbent assembly 160 can include a matrix of absorbent fibers, and more suitably cellulosic fluff, such as wood pulp fluff, and superabsorbent particles. One suitable pulp fluff is identified with the trade designation CR1654, commercially available from AbitibiBowater Inc. of Montreal, Quebec, Canada. As an alternative to wood pulp fluff, synthetic fibers, polymeric fibers, meltblown fibers, short cut homofil bicomponent synthetic fibers, or other natural fibers can be used. Suitable superabsorbent materials can be selected from natural, synthetic, and modified natural polymers and materials. The superabsorbent materials can be inorganic materials, such as silica gels, or organic compounds, such as crosslinked polymers, for example, sodium neutralized polyacrylic acid. Suitable superabsorbent materials are available from various commercial vendors, such as Evonik Stockhausen, LLC, Greensboro, N.C., U.S.A.; BASF Corporation, Florham Park, N.J., U.S.A.; and Nippon Shokubai, Chattanooga, Tenn., U.S.A., among others.

In one aspect, the absorbent assembly 160 can be stretchable so as not to inhibit the stretchability of other components to which the absorbent assembly can be adhered, such as the outer cover 162 and/or the bodyside liner 164. For example, the absorbent assembly 160 can include materials disclosed in U.S. Pat. Nos. 5,964,743, 5,645,542, 6,231,557, 6,362,389, and international patent application WO 03/051254, the contents of which are incorporated herein by reference to the extent that they are consistent (i.e., not in conflict) herewith.

In some aspects, a surge management layer (not shown) can be included in the training pants 12. The surge management layer can be positioned in the training pants 12 in a variety of locations as is known in the art. For example, the surge management layer can be proximate the absorbent assembly 160, for example between the absorbent assembly 160 and the bodyside liner 164, and attached to one or more components of the training pants 12 by methods known in the art, such as by adhesive, ultrasonic or thermal bonding. In addition, the surge management layer can be positioned in the training pants 12 relative to the leakage prevention element 20 in a variety of ways. For instance, the surge management layer can be disposed toward the liner 164 relative to the leakage prevention element 20, or the surge management layer can be disposed toward the absorbent assembly 160 relative to the leakage prevention element 20.

A surge management layer helps to decelerate and diffuse surges or gushes of liquid that can be rapidly introduced into the absorbent assembly 160. Desirably, the surge management layer can rapidly accept and temporarily hold the liquid prior to releasing the liquid into the storage or retention portions of the absorbent assembly 160. Examples of suitable surge management layers are described in U.S. Pat. No. 5,486,166 and U.S. Pat. No. 5,490,846, the contents of which are incorporated herein by reference to the extent that they are consistent (i.e., not in conflict) herewith.

The training pant 12 also includes a pair of side panels 166. Each side panel 166 is positioned transversely outward from the absorbent assembly 160 and bonded to the outer cover 162, the bodyside liner 164, or both using adhesives, thermal bonds, ultrasonic bonds or other suitable means.

The side panels 166 are desirably formed of an elastic material capable of stretching in a direction parallel to the transverse centerline 143 of the training pant 12. Further, the side panels 166 can also be formed of a gas permeable material, referred to as breathable material. The side panels 166 can, for instance, comprise a single layer of apertured film, a woven material, a nonwoven material or another suitable liquid permeable or liquid impermeable material. The side panels 166 can also comprise a laminate material, such as a stretch bonded laminate formed of a prestretched elastic meltblown inner layer sandwiched between and attached to a pair of spunbond polypropylene nonwoven webs having a basis weight of about 13.6 grams per square meter. Suitable elastic materials can be purchased from the Kraton Polymers of Houston, Tex., U.S.A. under the trade name KRATON.

In one aspect, the side panels 166 are formed of a laminated material including a prestretched elastic meltblown inner layer having a basis weight of about 18 grams per square meter (gsm) sandwiched between and stretch bonded to a pair of spunbond webs each having a basis weight of about 14.9 gsm. The spunbond webs comprise bicomponent fibers formed of about 50 weight percent polypropylene and about 50 weight percent polyethylene in a side-by-side configuration. Alternately, suitable elastic strands can be substituted for the elastic meltblown layer.

The training pant 12 can also include leg elastic members 168 and waist elastic members 170 that are bonded to the outer cover 162, the bodyside liner 164, or both to enhance fit and performance (FIG. 3). In particular, the leg elastic members 168 are operatively joined to the outer cover 162 along each side edge 134 through the crotch region 150. Also, the waist elastic members 170 are operatively joined to the outer cover 162 along the front and back end edges 130, 132. The elastic members 168, 170 can be bonded in place using adhesives, thermal bonds, ultrasonic bonds, stitching, or other suitable means. The elastic members 168, 170 can be stretch bonded to the outer cover 162, bonded in a relaxed state to a gathered portion of the outer cover 162, or a combination of the two. One suitable method for attaching the elastic members 168, 170 is disclosed in U.S. Pat. No. 4,639,949 issued Feb. 7, 1987, to Ales et al., the contents of which are incorporated herein by reference to the extent that they are consistent (i.e., not in conflict) herewith.

The training pant 12 can further include a pair of containment flaps (not shown) for inhibiting the lateral flow of body exudates. Containment flaps can be operatively attached to the training pant 12 in any suitable manner as is well known in the art. In particular, suitable constructions and arrangements for the containment flaps are generally well known to those skilled in the art and are described in U.S. Pat. No. 4,704,116 issued Nov. 3, 1987 to Enloe, the contents of which are incorporated herein by reference to the extent that they are consistent (i.e., not in conflict) herewith.

As noted previously, the leakage prevention element 20 is positioned and adapted to create a distinct physical sensation upon the absorbent article 10 approaching fullness. As the absorbent assembly 160 fills with urine or other body exudates, the urine or other body exudates wicks into the leakage prevention element 20 where the urine or other body exudates initiates a physical sensation that can be felt by the wearer of the absorbent article 10, thus helping to prevent leakage and alerting the wearer that a leak might soon occur.

In a number of aspects of the present disclosure illustrated in FIG. 3, the leakage prevention element 20 is positioned within the training pant 12 so that urine or other body exudates filling the absorbent assembly 160 contacts the leakage prevention element 20 prior to completely filling and eventually leaking from the training pant 12. Thus, the leakage prevention element 20 is disposed with or near the absorbent assembly 160 so that urine or other body exudates contacting the absorbent assembly will also eventually contact the leakage prevention element 20. Most desirably, the leakage prevention element 20 is disposed on the bodyside of the absorbent assembly 160 so as to be sandwiched between the absorbent assembly 160 and the bodyside liner 164. In this way, the physical sensation resulting from the leakage prevention element 20 is more easily noticed by the wearer. Generally, the closer to the perimeter 165 of the absorbent assembly 160 the leakage prevention element 20 is placed, the less time a wearer will have to change the article 10 before a leak can occur. The remaining time available to a wearer is also dependent on other factors including the nature of the bodily waste captured in the absorbent assembly 160.

In another aspect of the present disclosure (not shown), the leakage prevention element 20 can be located within the absorbent assembly 160 or beneath the absorbent assembly 160. The leakage prevention element 20 can also be positioned on the containment flaps, on the front or back waist region 152, 154, or in any other suitable position in the training pant 12, as long as fluid communication is provided between the absorbent assembly 160 and the leakage prevention element 20. In addition, leakage prevention elements 20 can be positioned in more than one location within the training pant 12. The leakage prevention element 20 can be maintained in position by bonding, using adhesives, ultrasonic bonds, or other suitable means.

One or more leakage prevention elements 20 can be disposed in the training pant 12. A pair of leakage prevention elements 20 can be positioned on opposite sides of the longitudinal centerline 141 and spaced apart from the intersection of the longitudinal and transverse centerlines 141, 143 along the transverse centerline 143. Similarly, a pair of leakage prevention elements 20 can be positioned on opposite sides of the transverse centerline 143 and spaced apart from the intersection of the longitudinal and transverse centerlines 141, 143 along the longitudinal centerline 141. In another aspect, leakage prevention elements 20 can be positioned at each of the points at which a centerline meets the perimeter 165 of the absorbent assembly 160. In still another aspect, the leakage prevention elements 20 can be positioned completely or partially along the entire absorbent assembly perimeter 165.

The position and/or structure of the leakage prevention elements 20 should be such that the leakage prevention elements 20 come in contact with urine or other bodily waste as the absorbent assembly 160 fills but prior to any leakage from the absorbent assembly 160. The leakage prevention elements 20 can be centered in the longitudinal direction 140. Alternatively, however, the leakage prevention elements 20 can be located off the transverse centerline 143 of the training pant 12. Likewise, the leakage prevention elements 20 can be centered in the transverse direction 142 or can be located off the longitudinal centerline 141 of the training pant 12.

As illustrated in FIG. 3, the leakage prevention element 20 can be positioned in the front 50 percent of the training pant 12. Because the training pant 12 is most likely to be in contact with the wearer in the region of the wearer's abdomen, the leakage prevention elements 20 are desirably positioned in the front waist region 152 and more particularly in the front one-third of the length of the training pant 12. Alternatively, leakage prevention elements 20 can be positioned in the back waist region 154, such as in the back one-third of the length of the training pant 12.

In other aspects of the present disclosure, the leakage prevention elements 20 can be positioned such that they are spaced apart from the absorbent assembly 160. For example, the leakage prevention elements 20 can be positioned between the liner 164 and the outer cover 162, but separated from the absorbent assembly 160. In such an arrangement, accommodation can be made to ensure urine or other bodily waste is transported from the absorbent assembly 160 to the leakage prevention elements 20. Such transport can be accomplished with the liner 164 or with a separate transport member.

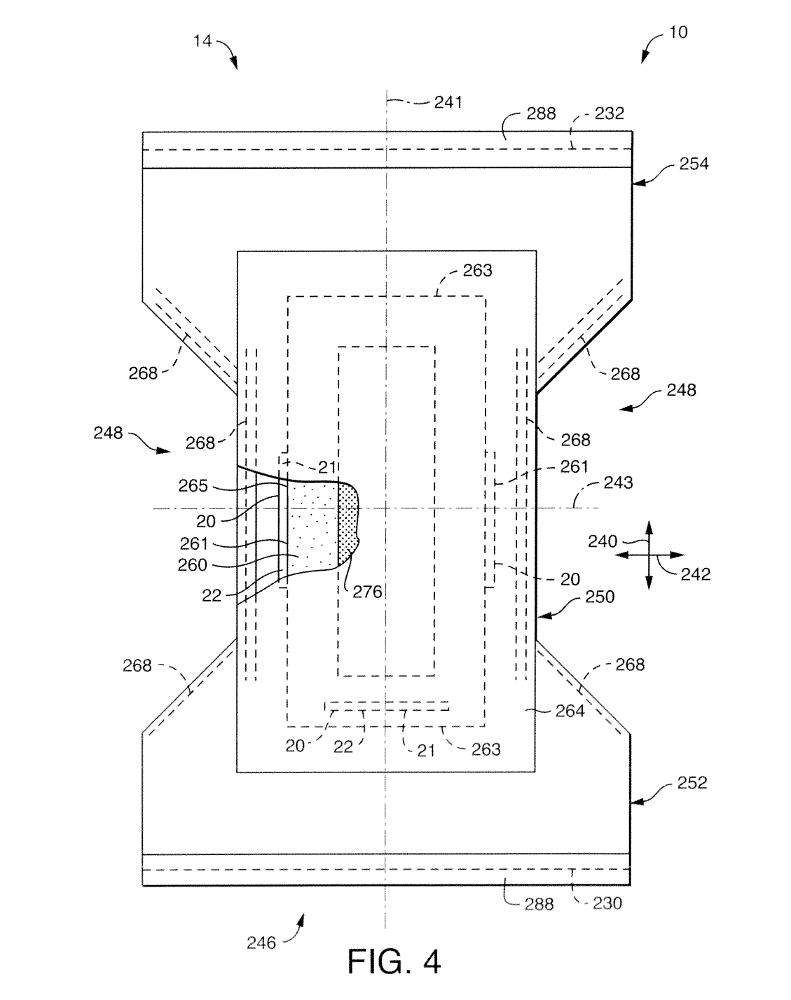

Referring to FIG. 4, a disposable pant-like absorbent adult undergarment 14 is depicted. The undergarment 14 is designed to be pulled up around a wearer's torso without having to first open the undergarment 14 to place it on a wearer's body. In FIG. 4, the undergarment 14 is shown in a flattened condition.

The undergarment 14 has a longitudinal direction 240 and a transverse direction 242, and includes a longitudinal centerline 241 and a transverse centerline 243. The undergarment 14 includes a front waist region 252, a back waist region 254 and a crotch region 250. The crotch region 250 joins the front waist region 252 to the back waist region 254. The front and back regions 252 and 254 can be stretchable. By “stretchable” it is meant that the regions 252 and 254 can be increased in size, for example lengthened, widened or extended in one or more dimensions by applying a force, such as by pulling. The crotch region 250 can be stretchable or non-stretchable but desirably is non-stretchable.

The front waist region 252 has a front end edge 230 and the back waist region 254 has a back end edge 232. The undergarment 14 can be folded approximately along the transverse centerline 243 such that the front end edge 230 aligns with the back end edge 232.

The front waist region 252 and the back waist region 254 are shown as being separate and discontinuous from one another although the front, back and crotch regions 252, 254, and 250 could be formed from a single piece of material, if desired. Desirably, the front waist region 252 is formed from a similar or identical material as the back waist region 254. The undergarment 14 has a bodyside liner 264 and an outer cover (not shown). The bodyside liner 264 will be in direct contact with the wearer's skin when the undergarment 14 is worn. The outer cover or garment-facing surface will be spaced away from the wearer's skin and will be adjacent to any outer clothing that the wearer can be wearing.

The front and back waist regions 252 and 254 can be constructed from various materials. The material can be a single layer or be a laminate of two or more layers. Spunbond is a material that works well for the front and back waist regions 252 and 254. Spunbond is a nonwoven material that is capable of being stretched at least a minimum amount. Spunbond is manufactured and sold by Kimberly-Clark Corporation of Neenah, Wis., U.S.A. Any other suitable material can also be used.

Other options for the front and back waist regions 252 and 254 can include two outer layers having a plurality of elastic strands sandwiched therebetween. The elastic strands can be formed from LYCRA brand elastic available from Invista of Wichita, Kans., U.S.A.

The undergarment 14 also includes an absorbent assembly 260 present in the crotch region 250. The absorbent assembly 260 can include a liquid pervious bodyside cover, a liquid-impervious outer cover, and an absorbent positioned therebetween. The absorbent in the absorbent assembly 260 can be formed from natural or synthetic materials. The absorbent can be made from cellulosic fibers, wood pulp, textile fibers or from other absorbent materials known to those skilled in the art. Superabsorbents, commonly in solid form and in the shape of small particles, granules, flakes, etc., can be mixed, combined, attached, printed or otherwise added to the absorbent material to increase the absorbent capacity of the absorbent. A surge layer 276 can also be optionally used, which is normally positioned between the bodyside liner 264 and the absorbent assembly 260. The surge layer 276 can function to rapidly acquire and temporarily retain body fluid, such as urine, before it can be absorbed into the absorbent. Desirably, the surge layer 276 is also capable of wicking body fluid lengthwise and/or widthwise across its surface as well as directing the body fluid downward in a z-direction, toward the absorbent.

The undergarment 14 further includes a waistband 288 secured to the front and back end edges 230 and 232. A portion of the waistband 288 overlaps each of the front and back waist regions 252 and 254 and projects outward therefrom. The waistband 288 can be described as extending outward from the front and back end edges 230 and 232 in a cantilevered configuration.

The waistband 288 can be constructed from almost any elastic material having stretch and retraction capabilities. A desirable nonwoven material from which the waistband 288 can be constructed is spunbond. The waistband 288 can be a laminate containing a first layer, a second layer and two or more elastic strands positioned therebetween. The outer two layers can be constructed or formed from a woven or a nonwoven material, a natural or synthetic material, an elastic film, a thermoplastic film, or from any other material known to those skilled in the art. The number of elastic strands positioned between the two outer layers can vary depending upon the width of the waistband 288. The elastic strands can be formed from LYCRA brand elastic available from Invista of Wichita, Kans., U.S.A.

The undergarment 14 further includes a pair of side seams (not shown) that function to join, bond and/or secure the front waist region 252 to the back waist region 254. The pair of side seams extends through the waistband 288 as well to form a unitary undergarment. By “unitary” it is meant that the undergarment 14 is designed to be stepped into by a wearer and the undergarment 14 is then pulled up along the wearer's legs and thighs and positioned around the wearer's torso. There is no need to first open a unitary undergarment before it is applied to a wearer's body. The unitary undergarment 14 has a longitudinal centerline 240, a waist opening 246, and a pair of leg openings 248.

The undergarment 14 further includes leg elastic members 268, each of which at least partially surrounds the pair of leg openings 248. The elastic members 268 can consist of one or more elastic strands. Each of the elastic members 268 can be formed as a continuous or a non-continuous member. In FIG. 4, each of the elastic members 268 is depicted as two separate and distinct members that are spaced apart from one another. However, a single elastic member 268, consisting of two or three elastic strands, could be employed that extend from one side seam to the opposite side seam.

The undergarment 14 can also include leg cuffs or leg flaps (not shown), each of which at least partially surrounds the pair of leg openings 248, to better seal the leg openings 248 against leakage. The leg cuffs can also include elastic members consisting of one or more elastic strands. Each of the elastic members can be formed as a continuous or a non-continuous member.

The absorbent assembly 260 can be in a variety of shapes and configurations as are known in the art, such as rectangular, hourglass shaped, l-shaped, and the like. The absorbent assembly 260 has opposed lateral edges 261 and opposed longitudinal ends 263. The lateral edges 261 and longitudinal ends 263 together make up the perimeter 265 of the absorbent assembly 260.

The leakage prevention element 20 is positioned within the adult garment 14 so that urine or other body exudates filling the absorbent assembly 260 contacts the leakage prevention element 20 prior to completely filling and eventually leaking from the adult garment 14. Thus, the leakage prevention element 20 is disposed with or near the absorbent assembly 260 so that urine or other body exudates contacting the absorbent assembly 260 will also eventually contact the leakage prevention element 20. Most desirably, the leakage prevention element 20 is disposed on the bodyside of the absorbent assembly 260 so as to be sandwiched between the absorbent assembly 260 and the bodyside liner 264. In this way, the physical sensation resulting from the leakage prevention element 20 is more easily noticed by the wearer.

In another aspect of the present disclosure (not shown), the leakage prevention element 20 can be located within the absorbent assembly 260 or beneath the absorbent assembly 260. The leakage prevention element 20 can also be positioned on the leg cuffs or containment flaps or in any other suitable position in the adult garment 14, as long as fluid communication is provided between the absorbent assembly 260 and the leakage prevention element 20. Leakage prevention elements 20 can be positioned on the flaps, positioned as a single web at the center of the flaps, slit with the flaps, or placed at the flap base. The leakage prevention element 20 can also be placed near the flap elastic members so that the substances are encapsulated in the flap material, not allowing the particle material to fall out. In addition, leakage prevention elements 20 can be positioned in more than one location within the adult garment 14. The leakage prevention element 20 can be maintained in position by bonding, using adhesives, ultrasonic bonds, or other suitable means.

One or more leakage prevention elements 20 can be disposed in the adult garment 14. As illustrated in FIG. 4, a pair of leakage prevention elements 20 is positioned on opposite sides of the longitudinal centerline 241 and spaced apart from the intersection of the longitudinal and transverse centerlines 241, 243 along the transverse centerline 243. Similarly, a pair of leakage prevention elements 20 can be positioned on opposite sides of the transverse centerline 243 and spaced apart from the intersection of the longitudinal and transverse centerlines 241, 243 along the longitudinal centerline 241 (not shown). In another aspect, leakage prevention elements 20 can be positioned at each of the points at which a centerline meets the perimeter 265 of the absorbent assembly 260 (not shown). In still another aspect, the leakage prevention elements 20 can be positioned completely or partially along the entire absorbent assembly perimeter 265 (not shown).

The position and/or structure of the leakage prevention elements 20 should be such that the leakage prevention elements 20 come in contact with urine or other bodily waste as the absorbent assembly 260 fills but prior to any leakage from the absorbent assembly 260. The leakage prevention elements 20 can be centered in the longitudinal direction 240. Alternatively, however, the leakage prevention elements 20 can be located off the transverse centerline 243 of the adult garment 14 (not shown). Likewise, the leakage prevention elements 20 can be centered in the transverse direction 242 or can be located off the longitudinal centerline 241 of the adult garment 14 (not shown).

The leakage prevention element 20 includes an active barrier 22. The size and shape of the active barrier 22 can vary widely. For example, an individual active barrier 22 can be rectangular and measure about 4 cm. (1.6 in) by about 7 cm. (2.8 in). Alternatively, the active barrier 22 can be in the form of strips (not shown) that extend over the full length or width of the absorbent article 10. As noted previously, the active barrier 22 is positioned and adapted to create a distinct physical sensation upon the absorbent article 10 approaching fullness. The physical sensation can be a pressure change such as from an expandable element or a physical contact from a moving element.

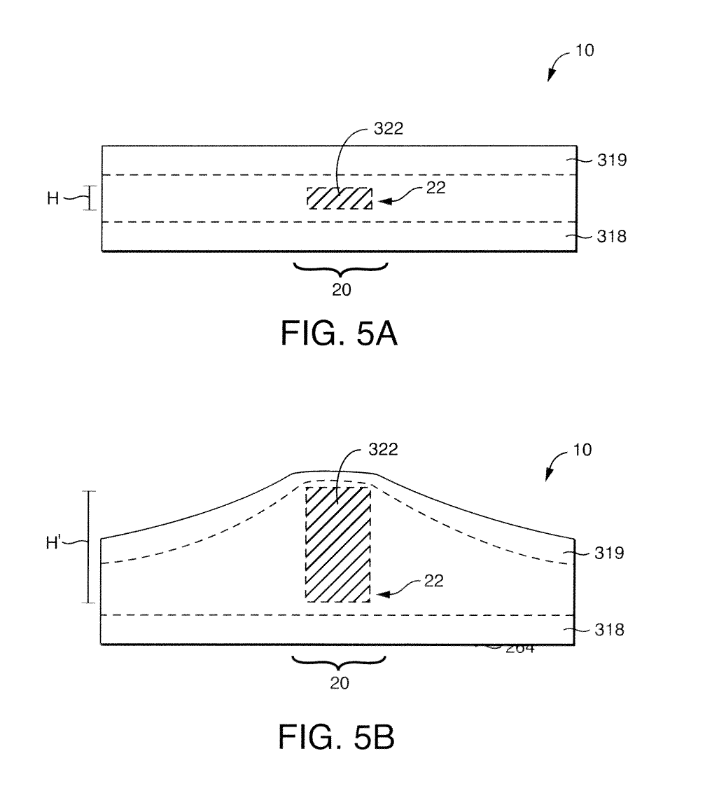

In one aspect of the present disclosure illustrated in FIGS. 5A and 5B, the leakage prevention element 20 is adapted to provide the wearer with an expanding or contracting dimensional change sensation using an active barrier 22. Dimensional change elements of this type are described in more detail in U.S. Pat. No. 5,649,914 to Glaug et al., the contents of which are incorporated herein by reference to the extent that they are consistent (i.e., not in conflict) herewith. The leakage prevention element 20 includes an active barrier 22 positioned on or within the absorbent article 10.

The active barrier 22 includes a material or materials that rapidly undergo a change in at least one dimension when exposed to an aqueous solution such as urine, menses, or other body exudate. The dimensional change is suitably either as an expansion to at least about 2 times a dry dimension or as a contraction to less than about one-half (½) of the dry dimension. In particular aspects, the dimensional change is either an expansion to at least about 5 times the dry dimension or a contraction to less than about one-fifth (⅕) of the dry dimension. In one example, the active barrier 22 has a wet height dimension that is at least about 5 times greater than its dry height dimension, and more desirably at least about 10 times greater for improved performance.

Another representative example is illustrated in FIGS. 5A and 5B. The active barrier 22 of height H is shown in FIG. 5A in its compressed or dry state, where element 18 generally refers to an absorbent assembly, and element 19 generally refers to a bodyside liner. The same active barrier 22 is shown in FIG. 5B in its wetted state, with a height of H′. The height dimension of the active barrier 22 is perpendicular to the plane formed by the longitudinal centerline 41, 141, 241 and transverse centerline 43, 143, 243 of the absorbent article 10 so that the dimensional change is noticeable to the wearer of the absorbent article 10. The other dimensions, the width and length, of the active barrier 22 can remain the same, expand, or contract when exposed to an aqueous solution.

In one particular aspect, the active barrier 22 includes a compressed cellulose sponge having a dry height of about 0.9 mm and a wet height of about 9.5 mm. The height dimensions are measured with the material under a compressive load of 0.2 pounds per square inch. The noncompressed axes of the material, that is the width and length, expand only about 7 percent from dry to wet states.

In one aspect of the present disclosure, the active barrier 22 is capable of expanding to at least about 5 times its dry height in 10 seconds, and more particularly to at least about 10 times its dry height in 3 seconds for improved performance.

Suitable materials for use in the active barrier 22 include expandable foams, compressed cellulose sponges, superabsorbents, or the like. Particularly, desirable expandable foams include those having open, large cell, reticulated structures. Examples of such expandable foams are available from 3M of Tonawanda, N.Y., U.S.A., and Industrial Commercial Supply Co. of Akron, Ohio, U.S.A. The material forming the dimensional change member 22 can be softened by mechanical means or other suitable techniques so as to be less noticeable until urination occurs. One such means that is effective with compressed cellulose sponge is to run the material through a set of meshed gears with the gap between the gears set so that the material is sufficiently scored to make it pliable.

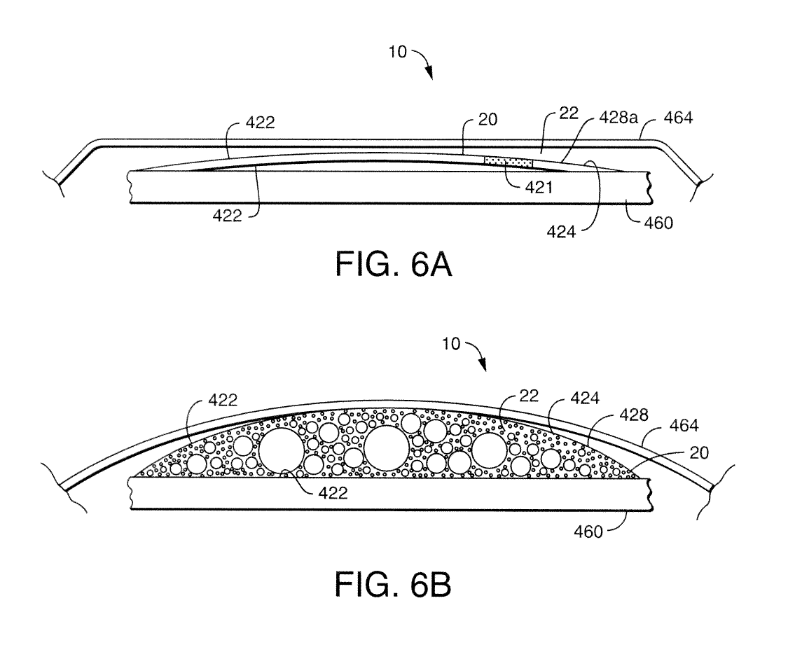

As illustrated in FIGS. 6A and 6B, the leakage prevention element 20 can include a substance that expands when the substance is contacted by urine or other body exudates, as is described in more detail below. That substance can be in the form of particles 421 captured between a pair of containment layers 422 or mixed into the absorbent assembly 460 or other material. The containment layers 422 form a container 424 to house and limit movement of the substance.

The leakage prevention element 20 can include an active barrier 22 constructed so that urine or other body exudates either enters the container 424 directly through the containment layers 422, is transported into the container 424 by the transport member, or both. Where urine or other body exudates is transported into the container 424, for example, the containment layers 422 can include a liquid impermeable material, such as a liquid impermeable film, a liquid impermeable nonwoven web, or the like. By either or both methods, urine or other body exudates will come into contact with the active barrier 22. The active barrier 22 will produce a physical sensation. As a result, the wearer will experience that physical sensation when the absorbent assembly 460 is approaching fullness to indicate to the wearer that potential leakage is imminent.

As illustrated in FIGS. 6A and 6B, the dimensional change member 428 can be a urine- or -other-body-exudates-permeable inflatable container 424 positioned between the bodyside liner 464 and the absorbent assembly 460. Dimensional change elements 428 of this type are described in more detail in U.S. Pat. No. 7,002,055 to Long et al., the contents of which are incorporated herein by reference to the extent that they are consistent (i.e., not in conflict) herewith. The inflatable container 424 can include a surfactant and a system that, upon wetting with urine or other body exudates, produces a gas, such as carbon dioxide. The gas produced upon wetting with urine or other body exudates interacts with the surfactant to produce a foam that inflates the container 424. The inflated container 424 pushes against the bodyside liner and causes the bodyside liner to press against the skin of the wearer to alert the wearer that the absorbent assembly 460 is nearing fullness.

The container 424 can be suitably formed from either woven or nonwoven substrates that are substantially liquid permeable to allow liquids, such as urine, to pass therethrough and contact the gas producing system and surfactant described herein. In one aspect, the inflatable liquid permeable container 424 can be formed from a 20 gsm spunbond nonwoven material available from Kimberly-Clark Corporation of Neenah, Wis., U.S.A. More particularly, a pair of opposed sheets of such material can be ultrasonically or otherwise bonded together along an edge margin about the periphery of the container 424 so as to seal the container 424. The container 424 can be either adhesively or thermally bonded to the absorbent assembly 460 and/or the bodyside liner 464 to stabilize the container 424 during use. The container 424 is sized, configured, and positioned in the absorbent article 10 in such a manner that the container 424 is free to expand or swell without substantial interference from other components of the absorbent article 10.

It should be understood that the container 424 could be fabricated from materials other than a spunbond nonwoven so long as at least a portion of the container 424 is sufficiently liquid permeable to permit liquid body exudates to permeate therethrough into the interior of the container 424 for contact with the gas producing systems and surfactants described herein.

As noted above, the permeable inflatable container 424 includes a system capable of generating a gas upon being wetted. The gas that is produced in the container 424 upon the wetting interacts with one or more surfactants, which are discussed below, and produces foam that inflates the container 424 and causes it to press the bodyside liner 464 against the skin of the wearer to alert the wearer that the absorbent assembly 460 is nearing fullness. FIG. 6B shows an absorbent assembly 460, a bodyside liner 464, and a permeable inflatable container 424, which is filled with foam. As shown in FIG. 6B, the inflated container 424 pushes against the bodyside liner 464 and distorts the bodyside liner 464. The distortion causes the bodyside liner 464 to press against the skin of the wearer to alert the wearer the nearing fullness of the absorbent assembly 460.

In one aspect, the system is capable of generating gas upon being wetted, which is located in the permeable inflatable container 424, includes at least one acid and at least one base. The acid and base react together upon being wetted to produce a gas that can be, for example, carbon dioxide gas. The exact gas produced by the gas producing system is not critical, so long as the gas produced is substantially non-harmful to the skin of the wearer.

In another aspect, the system capable of generating a gas upon being wetted includes a urine- or -other-body-exudates-soluble effervescent solid material produced in such a manner such that a pressurized gas is trapped within cells located in the solid material. When the solid material having pressurized gas-containing cells is contacted with urine or other body exudates, the solid material begins to dissolve and the pressurized gas is released from the cells during dissolution of the solid material. This gas can interact with the surfactant, also located in the permeable inflatable container 424, and produce a foam and bubbles that inflate the container 424 as described herein.

In this aspect, the soluble effervescent solid material can include a sugar compound such as a mono-saccharide, di-saccharide, or poly-saccharide that has been infused with a gas that is substantially non-reactive with human skin. Suitable gases for infusion into a solid material include, for example, carbon dioxide, air, nitrogen, argon, helium, other substantially inert gases, and combinations thereof. Specific examples of saccharides that can be used in accordance with the present disclosure include glucose, fructose, sucrose, lactose, maltose, dextrin, cyclodextrin, and the like, alone or in combination. Also, a mixture of sucrose with corn syrup (containing glucose, maltose, and dextrin) can be used in accordance with this aspect of the present disclosure to produce a gas-containing effervescent agent. Other examples of compounds that are capable of being prepared in such a manner as to trap pressurized gas in cells include, for example, water soluble compounds such as salts, alkali halides, and alkaline earth metal halides. Specific salts useful in the present disclosure include, for example, sodium chloride, potassium chloride, potassium bromide, lithium chloride, cesium chloride, and the like. Typically, the cells containing the pressurized gas have a diameter of from about 5 micrometers to about 100 micrometers.

The substantially non-reactive gas can be infused into the cells of the soluble solid material to produce an effervescent agent useful in the present disclosure by first heating the starting material, such as a sugar, in a small amount of water until the material is dissolved. After dissolution of the material, the water is evaporated off leaving the material in a molten state. The molten material is then gasified by introducing a suitable gas, such as carbon dioxide, at a superatmospheric pressure into a sealed vessel containing the molten material. The molten material is agitated during gasification to ensure intimate contact between the molten material and the gas. Pressures of, for example, between about 50 psig (340 kPa) and about 1000 psig (6890 kPa) can be utilized to infuse the gas into the molten material. After gas infusion, the molten material is allowed to solidify while maintained in the sealed vessel to produce an effervescent agent. A suitable procedure of producing a gas-containing solid material is fully set forth in U.S. Pat. No. 4,289,794 issued to Kleiner et al., the contents of which are incorporated herein by reference to the extent that they are consistent (i.e., not in conflict) herewith. The above procedure can produce solid effervescent agents containing cells of pressurized gas from about 50 psig (340 kPa) to about 900 psig (6200 kPa) that, when exposed to urine or other body exudates, allow the release of the trapped gas. This trapped gas, when released, can interact with the surfactant material in the container 424 described herein. The container 424 can suitably include from about 0.1 grams to about 15 grams of effervescent solid material containing a pressurized gas.

As noted above, the container 424 additionally includes a surfactant. The surfactant component located in the permeable inflatable container 424 is present as a foaming agent. When a gas, such as carbon dioxide, is produced from the gas generating system located in the container 424, the gas interacts with the surfactant and a bubble-filled foam is produced. These bubbles inflate the container 424 and cause it to swell and push against the bodyside liner 464 that, in turn, pushes against the skin of the wearer to alert the wearer to the nearing fullness of the absorbent assembly 460.

The surfactant used is not critical so long as it does not substantially irritate the skin upon contact. A wide variety of surfactants can be suitable for use in accordance with the present disclosure. For example, suitable surfactants include anionic surfactants, nonionic surfactants, amphoteric surfactants, cationic surfactants, and combinations thereof. Examples of suitable anionic surfactants include alkyl benzene sulfonates, alkyl sulfates, alkyl ether sulfates, sulfosuccinates, and combinations thereof. Examples of suitable nonionic surfactants include ethoxylated alcohols, fatty acid alkanolamides, ethoxylated alkanolamides, amine oxides, and combinations thereof. Examples of suitable amphoteric surfactants include alkyl betaines, amidobetaines, and combinations thereof. Examples of suitable cationic surfactants include alkylammonium halides. Generally, the container 424 will include from about 0.1 grams to about 15 grams of surfactant.

In one aspect of the present disclosure, the components included in the system capable of generating a gas, such as carbon dioxide, upon being wetted and/or the surfactant present in the permeable inflatable container 424 can be encapsulated in a urine- or -other-body-exudates-soluble shell material prior to introduction into the container 424. For example, if the system capable of generating a gas upon being wetted includes an acid and a base, the acid and the base can be separately encapsulated in a soluble encapsulation material to keep the components separated until wetted. Alternatively, the acid and base components can be encapsulated together if reactivity between the acid and the base in the absence of a liquid is not a concern. The surfactant can be separately encapsulated, or can be encapsulated with the acid and/or the base. Additionally, encapsulation can be used with gas-impregnated effervescent agents alone or in combination with the surfactant.

The shell material used for encapsulation can be suitably constructed of a material such that it will release the encapsulated material (i.e., the acid, base, effervescent agent and/or surfactant) upon contact with urine or other body exudates. The urine or other body exudates can cause the shell material to solubilize, disperse, swell, or disintegrate, or the shell material can be permeable such that it disintegrates or discharges the encapsulated material upon contact with urine or other body exudates. Suitable shell materials include cellulose-based polymeric materials (e.g., ethyl cellulose), carbohydrate-based materials (e.g., starches and sugars) and materials derived therefrom (e.g., dextrins and cyclodextrins) as well as other materials compatible with human tissues.

The shell thickness can vary depending upon the material encapsulated, and is generally manufactured to allow the encapsulated component to be covered by a thin layer of encapsulation material, which can be a monolayer or thicker laminate, or can be a composite layer. The layer should be thick enough to resist cracking or breaking of the shell during handling or shipping of the product or during wear that would result in breakage of the encapsulating material. The material should also be constructed such that humidity from atmospheric conditions during storage, shipment, or wear will not cause a breakdown of the microencapsulation layer.

In another aspect of the present disclosure, the leakage prevention element 20 can include an active barrier 22 that includes a liquid-shrinkable string of the type described in U.S. Patent Publication No. 2010/0152692, the contents of which are incorporated herein by reference to the extent that they are consistent (i.e., not in conflict) herewith. In this aspect, the active barrier(s) 22 present in the article 10 have the ability to remain flat before insult, thus increasing the level of comfort to the user. Upon liquid insult, the liquid-shrinkable string 524, a portion of which is positioned within the active barrier(s) 22, shrinks and pulls the active barrier(s) 22 inwards (i.e., toward the longitudinal and/or transverse centerlines of the article 10), causing the active barrier 22 to lift and activate to a desired shape, forming a closer body fit. With the closer body fit, liquid has a greater tendency to be maintained within the article 10, reducing the possibility of leakage.

The active barrier 22 includes a barrier structure member 528. The barrier structure member 528 can be formed from many of the types of materials. For example, the barrier structure member 528 can be formed from soft flexible material such as foam, fluff, gel, silicone, rubber, paper, nonwoven, film, or the like. For instance, absorbent material such as webs or laminates of absorbent material, with or without superabsorbent materials, can be suitable. Examples of suitable absorbent materials also include webs of cross-linked cellulosic fibers and meltblown webs. Alternatively, the barrier structure member 528 can be made from impervious materials. Examples of suitable nonabsorbent materials include thermoplastic polyethylene, polypropylene, synthetic foams, films or suitable blends of the types of materials described herein. In one particular aspect, a thermoplastic foam such as modified polyvinyl alcohol, polyethylene oxide, polypropylene oxide, ethylene oxide-propylene oxide copolymer, polyethylene glycol, polypropylene glycol, ethylene glycol-propylene glycol copolymer, modified polysaccharides, such as hydroxypropyl cellulose, methyl cellulose, methyl ethyl cellulose, polyethylene imine or combinations thereof can be used to make the barrier structure member 528. Suitable foams are described in U.S. patent application Ser. Nos. 11/117,864 to Zhang et al. and 11/027,306 to Radwanski et al., the contents of each of which are incorporated herein by reference to the extent that they are consistent (i.e., not in conflict) herewith. Other suitable foams are available commercially. For example, foams that retain bulk thickness after hydraulic needling (i.e., resilient foams) include RYNEL 562-B medical grade polyurethane and RYNEL 562-D medical grade polyurethane, both available from Rynel Inc. of Wiscasset, Me., U.S.A.

Other suitable foam layers include MINICELL STD crossed-linked polyethylene, available from Sekisui Voltek of Lawrence, Mass., U.S.A.; latex foams such as those described in U.S. Pat. No. 6,627,670 to Mork et al., the contents of which are incorporated herein by reference to the extent that they are consistent (i.e., not in conflict) herewith; High Internal Phase Emulsion (HIPE) foams such as those described in U.S. Pat. No. 5,260,345 to DesMarais et al., the contents of which are incorporated herein by reference to the extent that they are consistent (i.e., not in conflict) herewith; and extruded thermoplastic foams such as those described in U.S. patent application Ser. No. 10/729,881 filed Dec. 5, 2003 by Krueger et al. and U.S. Pat. No. 6,071,580 to Bland et al., the contents of both of which are incorporated herein by reference to the extent that they are consistent (i.e., not in conflict) herewith.

The active barrier 22 also includes a liquid-shrinkable string 524. The liquid-shrinkable string 524 can be in the form of yarn, fiber, filament, tape, film, nonwoven, laminate, and the like. In desirable aspects, the liquid-shrinkable string has a high ratio of length to diameter or width, though it need not be. The liquid-shrinkable string 524 is capable of activating the barrier structure member 528 to its desired shape upon sufficient fluid contact with the liquid-shrinkable string 524. The liquid-shrinkable string 524, upon exposure to urine, menstrual fluid or other bodily exudate, will shrink or shorten, thus increasing the tension in the liquid-shrinkable string 524. The liquid-shrinkable string 524 demonstrates shrinkage ability in both water (urine) and menstrual fluid. Shrinkage of at least about 10%, such as at least about 20%, or at least about 40%, or up to about 60% or more by length is suitable.

Suitable materials for the liquid-shrinkable string 524 include modified polyvinyl alcohol (PVA), modified cellulose fibers (e.g., cotton and rayon), such as carboxymethylated cotton, methylated cotton, ethylated cotton, hydroxyethylated cotton, sulfated cotton, sulfonated cotton, phosphated cotton, cationic cotton, amphoteric cotton, sodium acrylate-, acrylic acid-, acrylonitrile- or acrylamide-grafted cellulose fiber and crosslinked fiber thereof; wool or silk modified in the same manner as described above; modified synthetic fiber, such as a partially saponified acrylonitrile series of fiber and vinilon fiber that is partially esterified by maleic acid, carboxymethylcellulose and hydrolyzed acrylic fiber. In one particular aspect, a suitable modified PVA liquid shrinkable string can be obtained from Kuraray America, Inc. of Houston, Tex., U.S.A.

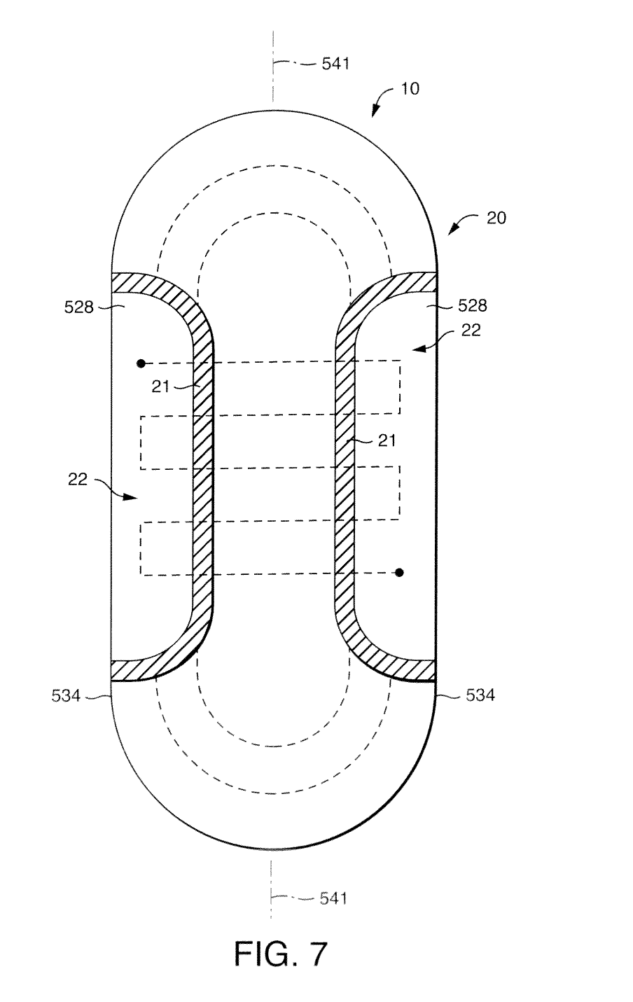

A first portion of the liquid-shrinkable string 524 is desirably attached to the barrier structure member 528 (e.g., attached to the surface of the barrier structure, or attached within the structure). In aspects where an additional barrier structure member 528 is present, a second portion of the liquid-shrinkable string 524 is attached to the additional barrier structure member 528. However, an additional string (i.e., a separate string) can additionally or alternatively be present in the one or more of the barrier structures. Additional, or multiple, strings can each include the same material, or they can include different materials. FIG. 7 shows a top view of an article 10 of the present disclosure having two barrier structure members 528 with four separate liquid-shrinkable strings 524 attached thereto, such as with adhesive or embossing, for example. Attachment of the liquid-shrinkable string 524 can occur through suitable bonding techniques including, but not limited to, stitching, adhesive bonds, cohesive bonds, thermal bonds, ultrasonic bonds, embossing, crimping, entangling, fusing, or the like, and combinations thereof. In some aspects, such bonding can occur over the entire length of the string. However, in other desirable aspects, at least one portion only of the string 524 can be bonded, such as one or more spot welds with adhesives, for example. In some aspects, it is desirable to anchor the ends of the string 524 into the article 10 or barrier structure member 528, while keeping the central length of the string 524 (e.g., the portion between one or more barrier structure members 528) free from bonds to provide improved shrinkage performance.

When menstrual fluid or other bodily exudate contacts one or more points of the liquid-shrinkable string 524, the total length of the liquid-shrinkable string 524 reduces, which creates a tension. The resulting tension pulls up the barrier structure member 528 away from the outer edge of the article 10 inwardly toward the centerline of the article 10. In the aspect illustrated in FIG. 7, two barrier structure members 528 are located on either side of the longitudinal-extending centerline 541 adjacent to the edges 534 of the article 10.

When the liquid-shrinkable strings 524 are contacted by an aqueous fluid, such as urine or menses, the liquid-shrinkable string 524 shrinks, which pulls on the barrier structure members 528 and lifts them.

In alternate aspects of the present disclosure, the leakage prevention element 20 can include heat-activatable, expandable structures that have the ability to stay flat before activation, hence providing ease of manufacture, ease of packaging, and discreet storage. Upon activation, the heat-activatable, expandable structures activate and expand to the desired shape, creating embossments, barriers, channels, and/or visual patterns on or between various layers of the absorbent article, thus allowing improved control of fluid to flow on or between the layers as well as improved visual appearance. Further description of these aspects is provided in U.S. patent application Ser. No. ______, the contents of all of which are incorporated herein by reference to the extent that they are consistent (i.e., not in conflict) herewith.

When the heat-activatable, expandable structure is heated, the heat-activatable, expandable structure expands to form a walled fluid containment region that prevents fluid leakage toward the front, rear, and/or sides. In use, the heat-activatable, expandable structure will inhibit flow of fluids from the central portion of the article 10 to the side and/or end edges of the article 10, thus inhibiting fluid leakage from the article 10. The heat-activatable, expandable structure can form any shape that is desired. For example, in one aspect, the heat-activatable, expandable structure has a shape that generally matches the shape of the article 10. In another aspect, the one or more heat-activatable, expandable structures can be disposed at one or more ends or sides of the article 10.

Suitable materials for the heat-activatable, expandable structure include heat-activatable, expandable inks, such as AQUAPUFF expandable inks from Polytex Environmental Inks Ltd. of Bronx, N.Y., U.S.A. AQUAPUFF expandable inks are expandable inks that react when exposed to heat to produce a gas by-product that causes the ink to expand or “puff up.” Suitable AQUAPUFF expandable inks include Puff Ink MW 4319 and MW 4404. Expandable inks can be applied using flexography, gravure, offset, inkjet (digital), curtain, knife and roll coating, silk screen, rotary screen, or any other suitable printing techniques. The printing of the inks can occur at a number of steps in the converting process, such as on an off-line printing step, or on-line during the product assembly process. Further, the ink can be printed at one process step, and expanded by heat activation at a downstream step.

The leakage prevention element 20 in each of the aspects described herein also includes a barrier highlight 21 associated with the active barrier 22. The barrier highlight 21 is configured to change appearance when wetted to highlight the action of the active barrier 22, making the dimension change of the active barrier 22 more perceivable to a wearer or caregiver.

In many cases, various elements of the articles described herein are white or otherwise uniform in their appearance and therefore difficult to differentiate. During use, even an active barrier 22 can be difficult for a wearer or caregiver to recognize because of little color differentiation from the surrounding materials. Providing a highlighted region in the form of a barrier highlight 21 that changes appearance under generally the same conditions that cause the active barrier 22 to change dimensions provides a visual confirmation of and therefore confidence in product functionality. Consumers are thereby aware of the functional elements (active barriers 22) in the products and have a greater awareness of and confidence in the product performing as designed.

The barrier highlight 21 generally overlies an active barrier 22 and is in fluid communication with the absorbent assembly 60, 160, 260, the active barrier 22, or both. In this use, “overlies” means that the barrier highlight 21 is disposed on or in a layer or structure between the outer cover and the inner surface such that the barrier highlight 21 can be seen from the inside surface of the article 10 before and/or after being wetted. The barrier highlight 21 can be disposed on or in the absorbent assembly 60, 160, 260, on or in the active barrier 22, on or in the liner 64, 164, 264, or on or in any other structure in the article 10. In addition, the barrier highlight 21 is disposed to generally coincide with the location of the active barrier 22. In various aspects, the barrier highlight 21 can overlap the active barrier 22 in the z-direction (perpendicular to the plane formed by the longitudinal and transverse directions 40, 140, 240 and 42, 142, 242). The barrier highlight 21 can partially or completely overlap the active barrier 22. In other aspects, the barrier highlight 21 can appear to adjoin or surround the active barrier 22. In general, “overlies” means that the barrier highlight 21 generally coincides with the active barrier 22 in the longitudinal and transverse directions 40, 140, 240 and 42, 142, 242, but not necessarily in the z-direction. Because of this arrangement, the barrier highlight 21 is enabled to change appearance when the active barrier 22 changes dimensions.

In various aspects of the present disclosure, the barrier highlight 21 is printed or otherwise disposed between the absorbent assembly 60, 160, 260 and the inner surface of the article 10 such that the barrier highlight 21 is visible from the inner surface of the article 10 either before being wetted, after being wetted, or both. The barrier highlight 21 includes a colorant as described herein. The colorant changes appearance when wetted. The colorant can change from one color to another, from one color to no color, or from no color to a color. Various portions of the barrier highlight(s) 21 need not be identical in their colorants or in the manner each of those portions changes appearance.

In one aspect of the present disclosure, the barrier highlight 21 is disposed on the liner 64, 164, 264, particularly on an absorbent-facing side of the liner 64, 164, 264. In other aspects, the barrier highlight 21 can be disposed on the absorbent assembly 60, 160, 260, on another structure in the article 10, or on an additional layer of material disposed between the liner 64, 164, 264 and the absorbent assembly 60, 160, 260.

The colorant used in or as each barrier highlight 21 can include any material that can indicate the desired property when an insult is received. The colorant can include a dye, such as bromocresol green; m-cresol purple; cresol red; chlorophenol red; bromothymol blue; bromopyrogallol red; bromoxylenol blue; methylene blue; bromophenol blue; monoazo dyes (such as acid alizarin violet N); monoazo pyrazoline dyes (such as acid yellow 34); diazo dyes (such as acid black 24); anthraquinone dyes (such as acid black 48); amphoteric anthraquinone dyes (such as acid blue 45); triphenylmethane dyes (such as acid fuchsin); phthalein type dyes (such as o-cresolphthalein); xanthene dyes (such as 2′,7′-dichlorofluorescein eosin B); heterocyclic acridine aromatics (such as acridine orange); diphenylmethane dyes (such as auramine O); triphenylmethane dyes (such as basic fuchsin); cationic thiazine dyes (such as azure C); cationic anthraquinone dyes (such as basic blue 47); phthalocyanine type dyes (such as copper phthalocyanine); quaternized phthalocyanine type dyes (such as alcec blue); cationic polymethine dyes (such as astrazon orange G); anthraquinone type (such as alizarin); the neutral complex dyes (such as azure A eosinate); the terpene type dyes (such as trans-beta-carotene); and so forth, as well as combinations including at least one of the foregoing dyes. The colorant 40 can also include leuco dyes and related chemistries such as those described in U.S. Patent Application Publication Nos. 2010/0114047 A1 and 2010/0030173 A1, and colorant chemistries such as those described in U.S. patent application Ser. No. 12/825,877, the contents of all of which are incorporated herein by reference to the extent that they are consistent (i.e., not in conflict) herewith. Desirably, the colorant(s) are U.S. FDA approved.

The colorant can be encapsulated in a material that is soluble in urine or menses. Suitable urine-, menses-, or water-soluble materials include, but are not limited to, cellulose-based polymeric materials (such as ethyl cellulose); carbohydrate-based materials (such as starches, sugars, and materials derived therefrom, e.g., dextrins and cyclodextrins); and so forth, as well as combinations including at least one of the foregoing materials. When the colorant is contacted by a liquid, the encapsulating material dissolves releasing the dye contained therein. The visual presence of the dye indicates that liquid is present.

The colorant can alternatively or additionally include one or more materials that can indicate the presence of a substance in or condition of the urine or menses. For example, pH levels in the urine or menses can be indicated by using methyl red, bromthymol blue, and phenolphthalein. The pH range 5 to 9 yields a color gradation from orange to yellow to green to blue. Any combination of the materials described herein can be encapsulated in a urine-, menses-, or water-soluble encapsulant. More than one colorant can be included in any single absorbent article 10.

In use, an article 10 of the present disclosure includes at least one active barrier 22 and a barrier highlight 21. The article 10 is donned by the wearer with or without the assistance of a caregiver. Upon insult with urine or other body exudates, the active barrier 22 changes dimension to both provide a barrier against leakage, and to provide a physical indication of the presence of liquid in the article 10. In addition, the barrier highlight 21 changes appearance to confirm the action of the active barrier 22 to reassure the wearer or caregiver that the active barrier 22 is working as intended.

Aspects of the disclosure have been described with reference to various specific and illustrative aspects and techniques. However, it should be understood that many variations and modifications can be made while remaining within the spirit and scope. Accordingly, this is intended to embrace all such alternatives, modifications and variations that fall within the spirit and scope of the appended claims. As various changes could be made in the above constructions and methods, without departing from the scope of the disclosure, it is intended that all matter contained in the above description and shown in the accompanying drawings shall be interpreted as illustrative and not in a limiting sense.

When introducing elements of the disclosure or the preferred aspect(s) thereof, the articles “a”, “an”, “the” and “said” are intended to mean that there are one or more of the elements. The terms “comprising,” “including,” and “having” are intended to be inclusive and mean that there can be additional elements other than the listed elements.

We claim:

1. An absorbent article for preventing leakage of a liquid insult, the article comprising:
an outer cover, a liner, and an absorbent assembly disposed therebetween, the liner having a body-facing surface;
an active barrier disposed between the liner and the outer cover, wherein the active barrier is in fluid communication with the absorbent assembly, and wherein the active barrier is adapted to change dimensions when wetted; and
a barrier highlight disposed to overlie the active barrier, wherein the barrier highlight is in fluid communication with the absorbent assembly or the active barrier, and wherein the barrier highlight is configured to change appearance when wetted.

2. The article of claim 1, wherein the barrier highlight becomes visible from the body-facing surface when the barrier highlight is wetted.

3. The article of claim 1, wherein the barrier highlight is visible from the body-facing surface when the barrier highlight is dry, and wherein the barrier highlight becomes substantially invisible from the body-facing surface when wetted.

4. The article of claim 1, wherein the barrier highlight is visible from the body-facing surface when the barrier highlight is both dry and wetted, and wherein the barrier highlight changes appearance from one color to a second color when the barrier highlight is wetted.

5. The article of claim 1, wherein the barrier highlight is disposed on the liner.

6. The article of claim 1, wherein the barrier highlight is configured to be responsive to wetness.

7. The article of claim 1, wherein the barrier highlight is configured to be responsive to pH.

8. The article of claim 1, wherein the barrier highlight is configured to be responsive to a component of the liquid insult.

9. The article of claim 1, wherein the active barrier is configured to be responsive to a first stimulus, wherein the barrier highlight is configured to be responsive to a second stimulus.

10. The article of claim 9, wherein the first stimulus is different from the second stimulus.

11. The article of claim 9, wherein the first stimulus is wetness, and the second stimulus is pH.

12. The article of claim 1, wherein the active barrier includes a swelling element.

13. The article of claim 1, wherein the active barrier includes a fluid-shrinkable element attached to a portion of the article.

14. The article of claim 1, the article further comprising transverse sides, wherein the active barrier is disposed adjacent a transverse side.

15. The article of claim 1, further comprising longitudinal ends, wherein the active barrier is disposed adjacent a longitudinal end.

16. The article of claim 1, further comprising an article perimeter, wherein the active barrier is disposed in a spaced-apart manner from the article perimeter.

17. The article of claim 1, wherein the article is a feminine pad.

18. The article of claim 1, wherein the article is a garment-like article including leg openings.

19. A feminine hygiene article for preventing leakage of a liquid insult, the article comprising:
a outer cover, a liner, and an absorbent assembly disposed therebetween, the liner having a body-facing surface, and the article including first and second transverse sides and a longitudinal direction;
a first longitudinally-extending active barrier disposed between the liner and the outer cover and adjacent the first transverse side, wherein the active barrier is in fluid communication with the absorbent assembly, and wherein the active barrier is configured to change dimensions when wetted; and
a barrier highlight disposed to overlie the active barrier, wherein the barrier highlight is in fluid communication with the absorbent assembly or the active barrier, and wherein the barrier highlight is configured to change appearance when wetted.

20. The article of claim 19, further comprising a second longitudinally-extending active barrier disposed between the liner and the outer cover and adjacent the second transverse side, wherein the active barrier is in fluid communication with the absorbent assembly, and wherein the active barrier is configured to change dimensions when wetted.

21. The article of claim 19, wherein the barrier highlight becomes visible from the body-facing surface when the barrier highlight is wetted.

22. The article of claim 19, wherein the barrier highlight is visible from the body-facing surface when the barrier highlight is both dry and wetted, and wherein the barrier highlight changes appearance from one color to a second color when the barrier highlight is wetted.
